(19) United States
(12) Patent Application Publication
(10) Pub. No.: US 2023/0100527 A1
(43) Pub. Date: Mar. 30, 2023

(54) MEDICAL USE VENTING FILTER

(71) Applicant: ENTEGRIS, INC., BILLERICA, MA (US)

(72) Inventors: Dongzhu Wu, Hangzhou (CN); Mei Feng He, Hangzhou (CN); Xi Bin Sun, Hanzhou City (CN); Kwok-Shun Cheng, Nashua, NH (US)

(21) Appl. No.: 17/954,236

(22) Filed: Sep. 27, 2022

(30) Foreign Application Priority Data
Sep. 29, 2021 (CN) 202111151805.2
Sep. 29, 2021 (CN) 202122378613.7

(51) Int. Cl. A61M 5/165; C08L 27/18; D04H 1/4318

(52) U.S. Cl. CPC A61M 5/165; C08L 27/18; D04H 1/4318; A61M 2005/1657; A61M 2005/1655; D10B 2509/00; D10B 2321/042

(57) ABSTRACT

The disclosure provides improved vent filters useful in single-use in-line transfusion systems. In a first aspect, the disclosure provides filter comprising (i) a layer comprising a fluoropolymer membrane and (ii) a layer comprising at least two air-permeable thermoplastic polymeric layers, the air-permeable thermoplastic polymeric layers comprised of a first polymeric layer and a second polymeric layer, wherein the first polymeric layer is in bonded contact with the fluoropolymer membrane, possesses a melting point of about 95° C. to about 180° C., and wherein the second polymeric layer is in bonded contact with the first polymeric layer and has a melting point of about 220° C. to about 265° C. These filters exhibit excellent bonding strength between the various layers while preserving a considerable amount of the original fluoropolymer membrane air flux.

### CROSS-REFERENCE TO RELATED APPLICATIONS

This application claims the benefit under 35 USC 119 of Chinese Invention Application No. 202111151805.2, filed Sep. 29, 2021, and Chinese Utility Model Application No. 202122378613.7, filed Sep. 29, 2021 the disclosure of each is hereby incorporated herein by reference in its entirety.

### TECHNICAL FIELD

The present disclosure relates generally to venting filters. In particular, it relates to venting filters useful in single-use in-line transfusion systems.

### BACKGROUND

Using disposable, i.e., single-use medical filters, such as in-line vent filter devices, while giving patients intravenous fluids is a common method in modern clinical treatment. Slow and incomplete venting during the use of disposable medical filters is a problem which has traditionally presented certain difficulties. If air in the infusion filter is not discharged in the beginning of the infusion process, the infusion cannot be performed normally. Additionally, if the air in the infusion filter is not effectively discharged during the infusion process, air potentially enters the patient's blood stream with a pulmonary embolism as a potential adverse outcome.

Traditionally, venting membranes on a single-use medical filter are generally a composite structure of polyester (e.g., poly(ethylene terephthalate)) nonwoven fibers and poly(tetrafluoroethylene) microporous membranes, prepared via a lamination process. Because the filter is in direct contact with the transfusion liquid, in order to avoid extractible materials, adhesives are not used during lamination. During a hot lamination process, it is inherently difficult to balance necessary and desired air permeability of the resulting laminated composite filter versus bonding strength between the polyester non-woven and the poly(tetrafluoroethylene) membrane. Often, when high temperatures and/or compression are utilized, the resulting laminate will suffer from low air permeability, i.e., high air flux loss. Thus, there remains a continuing need for such venting membranes having effective lamination, while also exhibiting adequate air flux rates.

### SUMMARY

The disclosure provides improved vent filters useful in single-use in-line transfusion systems. In a first aspect, the disclosure provides a filter comprising
(i) a layer comprising a fluoropolymer membrane and
(ii) a layer comprising at least two air-permeable thermoplastic polymeric layers, the air-permeable thermoplastic polymeric layers comprised of a first polymeric layer and a second polymeric layer,
wherein the first polymeric layer is in bonded contact with the fluoropolymer membrane and possesses a melting point of about 95° C. to about 180° C., and wherein the second polymeric layer is in bonded contact with the first polymeric layer and has a melting point of about 220° C. to about 265° C.

In certain embodiments, the air permeable thermoplastic polymeric layers can be in the same or different polymer class and are chosen from thermoplastic polymers such as polyesters and polyolefins. Other thermoplastic polymers can also be utilized, provided they possess the necessary melting temperatures as stated herein. Advantageously, these air permeable thermoplastic polymeric layers are comprised of woven or nonwoven fibrous structures. The air-permeable thermoplastic polymeric layers are advantageously comprised of materials which are compatible with and thus capable of thermal bonding to the housing of the filter, which is often comprised of acrylonitrile butadiene styrene copolymer (ABS), acrylonitrile-styrene copolymer (AS), or methyl methacrylate-acrylonitrile-butadiene-styrene copolymers (MABS).

In one embodiment, by utilizing a polyester layer component which possesses at least two layers, one of which is a lower-melting polyester and one of which is comprised of a higher-melting polyester, the resulting laminate provides a good balance of high (remaining) air flux along with good structural integrity, while also obviating a need for an adhesive or other means to effect proper lamination of the poly(tetrafluoroethylene) (PTFE) membrane layer with the structural polymer, for example a polyester such as poly(ethylene terephthalate) (PET).

In one embodiment, the lower-melting polymer layer is a poly(ethylene terephthalate) having a melting temperature of about 95° C. to about 180° C., or about 150° C. to 180° C. As used herein, the term “poly(ethylene terephthalate)” refers to polyesters comprised primarily of residues of terephthalic acid and ethylene glycol, but also encompasses such polymers where the diacid and/or glycol is other than terephthalic acid and ethylene glycol. Such modification to the polymer comprising the air permeable (e.g., woven or nonwoven) materials is well-known to those skilled and the art and can be designed for desired physical properties such as elastic modulus, melting temperature, compatibility with other polymers, etc.

This lower-melting temperature polyester can be thermally fused with a higher-melting polyester to form a bilayer structure which effectively serves as a support layer for the fluoropolymer (e.g., poly(tetrafluoroethylene) or “PTFE”) layer, while also maintaining sufficient air flux so that the resulting composite filter can effectively serve as a vent filter. Such bilayer materials are commercially available from Asahi Kasei, product designation C5030. In this C5030 product, the lower melting layer is a PET having a melting point of about 150° to 180° C. and a softening temperature of about 125° C.; the higher melting layer is a PET having a melting point of about 260° C.

By pre-heating the polyester substrate layer(s) and the microporous fluoropolymer layer prior to introduction to the laminating machine, the lower-melting point polyester layer can be fully melted, thus enabling thermal welding (i.e., bonding) with the PTFE layer, and on the other hand, the chains or chain segments on the poly(tetrafluoroethylene) can be effectively “stretched” before they are fixed during a compression stage, when the layers are laminated together under raised heat and compression. We have found that by applying an appropriate compression force under a certain (elongation) tension, we can reduce the shrinkage of the poly(tetrafluoroethylene) layer and thus maintain high membrane porosity, thereby greatly reducing the air flux loss of the resulting composite filter structure. The resulting composite filter exhibits excellent bonding strength while preserving a considerable amount of the original fluoropolymer membrane air flux at the same time. In general, the air flux of the starting fluoropolymer membrane was about 0.25 liters/minute and the air flux loss rate of the composite membrane, i.e., after lamination, was only about 30% to about 50%.

### BRIEF DESCRIPTION OF THE DRAWINGS

FIG. 1 is a depiction of an exemplary lamination operation, illustrating one aspect of the disclosure, wherein a PTFE microporous membrane is laminated to a polyester nonwoven material, the material comprising at least two layers, wherein the layer which ultimately comes into bonded contact with the PTFE microporous membrane is a relatively lower melting point polyester.

FIG. 2 is a depiction of a cross-section of an exemplary composite filter structure of the disclosure. The PTFE microporous membrane (9) is in bonded contact with a polyester nonwoven layer (10), which is in turn in contact with a polyester nonwoven layer (11). As described herein, the polyester nonwoven layer (10) is a lower melting point polyester than the polyester nonwoven layer (11).

FIG. 3 is a depiction of the structure and operation of a typical single use in-line infusion vent filter device. The polyester/PTFE composite membrane is bonded to the structure via the polyester layer, while the PTFE layer faces downward and contacts the liquid being infused to the patient. The polyester layer is typically hot-welded to the in-line infusion device structure. The directional flow of the infusion liquid is depicted from left to right, with exhausted air flowing upwards.

### DETAILED DESCRIPTION

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “about” generally refers to a range of numbers that is considered equivalent to the recited value (e.g., having the same function or result). In many instances, the term “about” may include numbers that are rounded to the nearest significant figure.

Numerical ranges expressed using endpoints include all numbers subsumed within that range (e.g., 1 to 5 includes 1, 1.5, 2, 2.75, 3, 3.80, 4 and 5).

In a first aspect, the disclosure provides a filter comprising (i) a layer comprising a fluoropolymer membrane and (ii) a layer comprising at least two air-permeable thermoplastic polymeric layers, the air-permeable thermoplastic polymeric layers comprised of a first polymeric layer and a second polymeric layer, wherein the first polymeric layer, in bonded contact with the fluoropolymer membrane, possesses a melting point from about 95° C. to about 180° C., about 95° C. to about 170° C., about 95° C. to about 160° C., about 95° C. to about 150° C., about 95° C. to about 140° C., about 95° C. to about 130° C., about 110° C. to about 170° C., about 110° C. to about 160° C., about 110° C. to about 150° C., about 110° C. to about 140° C., about 110° C. to about 130° C., about 125° C. to about 170° C., about 125° C. to about 160° C., about 125° C. to about 150° C., about 150° C. to about 180° C., about 150° C. to about 170° C., and all ranges and subranges therebetween. In some embodiments, the second polymeric layer is in bonded contact with the first polymeric layer and has a melting point from about 220° C. to about 265° C., about 220° C. to about 260° C., about 220° C. to about 255° C., about 220° C. to about 250° C., about 230° C. to about 265° C., about 230° C. to about 260° C., about 230° C. to about 255° C., about 230° C. to about 250° C., about 240° C. to about 265° C., about 240° C. to about 260° C., about 240° C. to about 255° C., about 240° C. to about 250° C., and all ranges and subranges therebetween.

In certain embodiments, the first and second polyester layers are comprised from about 70 to about 100 weight percent, about 70 to about 95 weight percent, about 70 to about 90 weight percent, about 80 to about 100 weight percent, about 70 to about 90 weight percent, about 90 to 100 weight percent of poly(ethylene terephthalate). In another embodiment, the melting point of the first polyester is about 150° to about 180° C. In another embodiment, the melting point of the second polyester layer is about 260° C.

Advantageously, the bonding strength exhibited by the laminated filter structure is greater than or equal to about 0.17 MPa (Mega Pascals), greater than or equal to about 0.2 MPa, greater than or equal to about 0.25 MPa, greater than or equal to about 0.3 MPa, greater than or equal to about 0.35 MPa, greater than or equal to about 0.4 MPa, greater than or equal to about 0.45 MPa, greater than or equal to about 0.5 MPa, greater than or equal to about 0.55 MPa, or greater than or equal to about 0.6 MPa.

In some embodiments, the filter exhibits an air flux from about 0.125 to about 0.210 liters/minute, about 0.125 to about 0.200 liters/minute, about 0.125 to about 0.175 liters/minute, about 0.125 to about 0.150 liters/minute, from about 0.150 to about 0.210 liters/minute, about 0.150 to about 0.200 liters/minute, about 0.150 to about 0.175 liters/minute, from about 0.175 to about 0.210 liters/minute, about 0.175 to about 0.200 liters/minute, and all ranges and subranges therebetween. In some embodiments, the filter exhibits an air flux in any of the above ranges in combination with a bonding strength of greater than or equal to about 0.2 MPa, greater than or equal to about 0.25 MPa, greater than or equal to about 0.3 MPa, greater than or equal to about 0.35 MPa, greater than or equal to about 0.4 MPa, greater than or equal to about 0.45 MPa, greater than or equal to about 0.5 MPa, greater than or equal to about 0.55 MPa, or greater than or equal to about 0.6 MPa.

In one embodiment, the filter exhibits an air flux from about 0.125 to about 0.175 liters/minute and a bonding strength of greater than or equal to about 0.35 MPa. In another embodiment, the filter exhibits an air flux from about 0.175 to about 0.210 liters/minute and a bonding strength of greater than or equal to about 0.2 MPa.

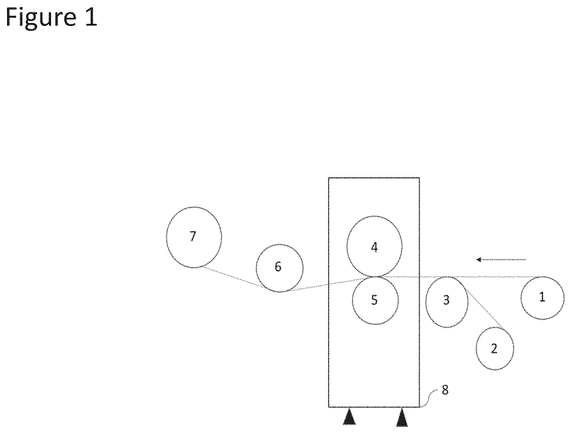

Referring to FIG. 1, in one embodiment, a poly(tetrafluoroethylene) (PTFE) microporous membrane is located on a roll (1) and fed to a pre-heating roller (3) before being fed into the laminating machine (8). Similarly, a polyester nonwoven material, for example, a combined lower-melting and higher-melting two-layer nonwoven material (such as the Asahi Kasai C5030 material, referred to above) is located on a roll (2) and is then is fed to the pre-heating roller (3), with the lower melting polyester layer facing upwards so that it will come into contact with the PTFE membrane layer. The preheating roller (3) is generally operated at a temperature of about 50° to 260° C., or about 100° to about 230° C. In order to effect lamination of the lower-melting layer of the two-layer material to the PTFE layer, and in order to form the desired composite, the two-layer polyester nonwoven material is advantageously fed to the pre-heating roller beneath the PTFE layer as shown, and then transmitted to the laminating machine (8), which contains the heating and compression rollers (4) and (5). Rollers (4) and (5) are generally disposed such that the compression pressure exerted on the combined layers is about 0.05 to about 0.4 MPa, about 0.05 to about 0.3 MPa, about 0.05 to about 0.2 MPa, about 0.1 to about 0.4 MPa, about 0.1 to about 0.3 MPa, about 0.1 to about 0.2 MPa, about 0.2 to about 0.4 MPa, or about 0.2 to about 0.3 MPa and the (elongation) tension placed on the combined layers of about 0.05 to about 0.3 Newtons (N), about 0.05 to about 0.2 N, about 0.1 to about 0.3 N, or about 0.1 to about 0.2 N. The temperature of Rollers (4) and (5) is generally from about 140° C. and 210° C., about 140° C. and 200° C., about 140° C. and 175° C., about 150° C. and 210° C., about 150° C. and 200° C., about 150° C. and 175° C., about 160° C. and 210° C., about 160° C. and 200° C., about 160° C. and 175° C., about 175° C. and 210° C., about 175° C. and 200° C., and all ranges and subranges therebetween. Residence time within the laminating machine (8) is advantageously only that time necessary to effect the desired lamination, which is generally about 1 to about 60 seconds, but can be determined empirically given the choice of PTFE, polyester layer(s), and their concomitant physical properties. Following this heating and compression step, the composite filter material is oriented via use of roller (6) and then collected on a storage roller (7). The heating rollers referred to herein may be constructed of any suitable material, such as stainless steel, PTFE-coated rollers, etc. The heating and compression rollers (4) and (5) referred to herein may be constructed of any suitable material, such as stainless steel, rubber, etc.

Accordingly, in another aspect, the disclosure provides a process for laminating (i) a fluoropolymer membrane and (ii) a bonded layer comprising at least two air-permeable thermoplastic polymeric layers, the air-permeable thermoplastic polymeric layers comprised of a first polymeric layer having a melting point of about 95° to about 180° C., and a second polymeric layer having a melting point of about 220° to about 265° C., wherein the first polymeric layer is in bonded contact with the fluoropolymer membrane and forms a composite fluoropolymer-thermoplastic polymeric filter structure, which comprises:
a. applying the bonded layer to a fluoropolymer membrane, thereby contacting the first polymeric layer of the bonded layer with the fluoropolymer membrane, on a surface having a temperature of about 50° to about 260° C. to form a combined bonded layer and fluoropolymer structure; and then
b. subjecting the combined bonded layer and fluoropolymer structure to a temperature of about 140° C. to about 210° C., while compressing at a pressure of about 0.05 to about 0.4 MPa, and while exerting an elongation tension on the combined bonded layer and fluoropolymer structure of about 0.05 to about 0.3 Newtons;
c. and cooling the resulting fluoropolymer-thermoplastic polymeric composite filter structure.

Once this lamination process is completed, the resulting fluoropolymer-thermoplastic polymeric composite structure can be actively cooled or merely allowed to cool to room temperature (step c.).

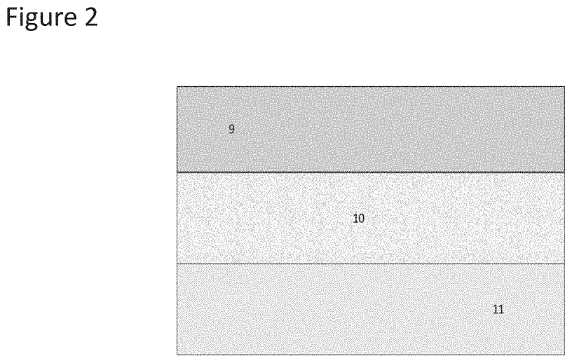

Referring to FIG. 2, the composite filter structure will be comprised of, for example, one poly(tetrafluoroethylene) (PTFE) layer (9), in bonded contact with a lower-melting polyester layer (10), which is in turn in bonded contact with a higher-melting polyester layer (11). The latter serves to provide adequate structural integrity for the overall filter structure.

The fluoropolymer membrane material (e.g., PTFE) useful in the disclosure are known microporous membrane structures and are commercially-available in many variations and can be obtained or prepared according to desired performance criteria. PTFE membranes are inherently hydrophobic and lipophobic and in one embodiment exhibit an air flux of about 0.25 to about 0.3 liters/minute, a thickness of about 50 to about 60 μm, and a bubble point of greater than about 0.17 MPa in ethyl alcohol.

The polyester nonwoven materials useful in the disclosure are widely available commercially. Such polyesters can be obtained having desired melting points for use as described herein, and include Product Type C5030, available from Asahi Kasei Corporation and sold under the marks Precis™ and ELTAS™, along with product type MB-12D, available from Mitsubishi Paper Mills Limited.

Suitable fluoropolymers effective as hydrophobic venting membranes are commercially available. Exemplary PTFE membranes include those sold by Donaldson Company under the Tetratex® mark, for example product designations TX1302, TX1320, and TX1333. The thickness of such membranes is in one embodiment about 50 to about 70 μm.

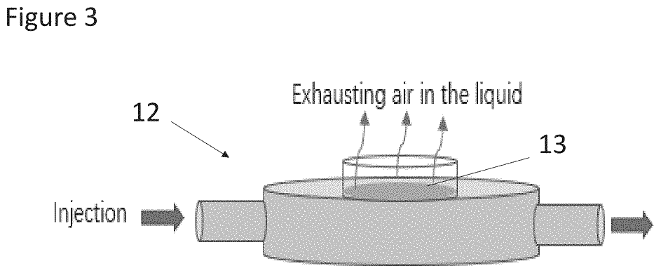

Any of the filters described above can be an in-line vent filter device as shown for example in FIG. 3. The in-line vent filter device 12 can have any of the composite membranes 13 described herein bonded to the in-line vent filter structure via the layer comprising at least two air-permeable thermoplastic layers, such as for example tow polyester nonwoven layers, while the fluoropolymer layer faces downward and contacts the liquid being infused to the patient. The composite membrane 13 is typically hot-welded to the in-line vent filter device structure. An infusion liquid is injected into the in-line vent filter device and is shown as flowing from left to right through the device and air filtered out through the composite membrane 13 exits through the top of the device 12.

### EXAMPLES

Test Methodology:

Bonding Strength Test:

The composite membrane with a diameter of 10 mm is placed in the membrane holder with the PTFE side facing upwards. Water pressure is applied from the downstream side starting from 0.15 MPa with an increasing rate of 0.02 MPa/15 seconds. After 15 seconds under pressure, we observe whether there are air bubbles on the membrane surface (the membrane and the support are layered). If there are no bulges or bumps, it means that the bonding strength is qualified at this strength. Then, the pressure is continued to increase until a bulge is observed during 15 seconds of pressure holding. Once a bulge is observed, the composite membrane is considered to be delaminated.

Air Flux Measurement:

The composite membrane with a diameter of 8 mm is placed in the membrane holder. Then the air flux (L/min) is measured at a pressure of 10 KPa as the passage rate of air at a rate of liters per minute. A Rotameter flowmeter was utilized to measure the air flow.

Air Flux Loss Rate:

The air flux loss rate (AFLR) is determined according to the following equation:

AFLR=(PTFE air flux-Composite membrane air flux)/(PTFE air flux)×100%.

A lamination operation as shown in FIG. 1 was utilized in performing the examples set forth below and reference to roller numbers refers to the corresponding rollers in FIG. 1.

### Example 1

Set the temperature of Roller-3 to 150° C., the temperature of Roller-4 to 200° C., the lamination pressure between Rollers-4 and 5 to 0.2 MPa, and the temperature of Roller 6 to room temperature, respectively. After the temperatures are stabilized, the PET non-woven fabric and the PTFE film are sequentially entered into the Roller-3, the Rollers-4 and 5, and the Roller 6 at a speed of 2 meters/minute under 0.1N (Newtons) tension, and the resultant membrane is shown in Example 1 in Table 1.

### Comparative Example 1

Set the temperature of Roller-3 to room temperature, the temperature of Roller-4 to 200° C., the lamination pressure between Rollers-4 and 5 to 0.2 MPa, and the temperature of Roller 6 to room temperature, respectively. After the temperatures are stabilized, the PET non-woven fabric and the PTFE film are sequentially entered into the Roller-3, the Rollers-4 and 5 and the Roller 6 at a speed of 2 m/min under 0.1N tension, and the resultant membrane is shown in Comparative Example 1.

### Comparative Example 2

Set the temperature of Roller-3 to room temperature, the temperature of Roller-4 to 220° C., the lamination pressure between Rollers-4 and 5 to 0.1 MPa, and the temperature of Roller 6 to room temperature, respectively. After the temperatures are stabilized, the PET non-woven fabric and the PTFE film are sequentially entered into the Roller-3, the Rollers-4 and 5 and the Roller 6 at a speed of 1 m/min under 0.25N tension, and the resultant membrane is shown in Comparative Example 2

### Comparative Example 3

Set the temperature of Roller-3 to room temperature, the temperature of Roller-4 to 180° C., the lamination pressure between Rollers-4 and 5 to 0.3 MPa, and the temperature of Roller 6 to room temperature, respectively. After the temperatures are stabilized, the PET non-woven fabric and the PTFE film are sequentially entered into the Roller-3, the Rollers-4 and 5 and the Roller 6 at a speed of 1.5 m/min under 0.2N tension, and the resultant membrane is shown in Comparative Example 3.

TABLE 1
Data Comparison of Different PET/PTFE Composite Membranes

| | Bonding | Air Flux | Air Flux |
|---|---|---|---|
| | Strength | (Liters | Loss |
| Sample | (MPa) | per minute) | (%) |
| Single PTFE membrane | (not applicable) | 0.25 | (not applicable) |
| Example 1 | >0.5* | 0.14 | 44% |
| Comparative Example 1 | 0.35 | 0.07 | 80% |
| Comparative Example 2 | >0.5 | 0.1 | 60% |
| Comparative Example 3 | 0.08* | 0.13 | 48% |

*The membrane passed the bonding strength test at 0.5 MPa and was not continued beyond 0.5 MPa.

This data shows the improvement in bonding strength due to the pre-heating step utilized in Example 1, while minimizing air flux loss during lamination.

Aspects

In a first aspect, the disclosure provides a filter comprising
(i) a layer comprising a fluoropolymer membrane and
(ii) a layer comprising at least two air-permeable thermoplastic polymeric layers, the air-permeable thermoplastic polymeric layers comprised of a first polymeric layer and a second polymeric layer,
wherein the first polymeric layer is in bonded contact with the fluoropolymer membrane and possesses a melting point of about 95° C. to about 180° C., and wherein the second polymeric layer is in bonded contact with the first polymeric layer and has a melting point of about 220° C. to about 265° C.

In a second aspect, the disclosure provides the filter of the first aspect, wherein the filter exhibits an air flux from about 0.125 to about 0.175 liters/minute and a bonding strength of greater than or equal to about 0.35 MPa.

In a third aspect, the disclosure provides the filter of the first aspect, wherein the filter exhibits an air flux from about 0.175 to about 0.210 liters/minute and a bonding strength of greater than or equal to about 0.2 MPa.

In a fourth aspect, the disclosure provides the filter of any one of the first, second, or third aspects, wherein the air-permeable thermoplastic polymeric layers are comprised of nonwoven fibers.

In a fifth aspect, the disclosure provides the filter of any one of the first through fourth aspects, wherein the fluoropolymer membrane is comprised of poly(tetrafluoroethylene).

In a sixth aspect, the disclosure provides the filter of any one of the first through the fifth aspects, wherein the thermoplastic polymeric layers are comprised of polymers chosen from polyesters and polyolefins.

In a seventh aspect, the disclosure provides a filter comprising
(i) a layer comprising at least one poly(tetrafluoroethylene) membrane and
(ii) a layer comprising at least two polyester nonwoven layers, the polyester nonwoven layers comprised of a first polyester layer and a second polyester layer, wherein the first polyester layer is in bonded contact with the poly(tetrafluoroethylene) membrane and possesses a melting point from about 95° C. to about 180° C., and wherein the second polyester layer is in bonded contact with the first polyester layer and has a melting point from about 220° C. to about 265° C.

In an eighth aspect, the disclosure provides the filter of the seventh aspect, wherein the filter exhibits an air flux from about 0.125 to about 0.175 liters/minute and a bonding strength of greater than or equal to about 0.35 MPa.

In a ninth aspect, the disclosure provides the filter of the seventh aspect, wherein the first and second polyester layers are comprised from about 70 to about 100 weight percent of poly(ethylene terephthalate).

In a tenth aspect, the disclosure provides the filter of the seventh aspect, wherein the melting point of the first polyester is from about 150 to about 180° C.

In an eleventh aspect, the disclosure provides a process for laminating (i) a fluoropolymer membrane and (ii) a bonded layer comprising at least two air-permeable thermoplastic polymeric layers, the air-permeable thermoplastic polymeric layers comprised of a first polymeric layer having a melting point from about 95° to about 180° C., and a second polymeric layer having a melting point from about 220° to about 265° C., wherein the first polymeric layer is in bonded contact with the fluoropolymer membrane and forms a composite fluoropolymer-thermoplastic polymeric filter structure, which comprises:
a. applying the bonded layer to a fluoropolymer membrane, thereby contacting the first polymeric layer of the bonded layer with the fluoropolymer membrane, on a surface having a temperature from about 50° to about 260° C. to form a combined bonded layer and fluoropolymer structure; and then
b. subjecting the combined bonded layer and fluoropolymer structure to a temperature from about 140° C. to about 210° C., while compressing at a pressure from about 0.05 to about 0.4 MPa, and while exerting an elongation tension on the combined bonded layer and fluoropolymer structure from about 0.05 to about 0.3 Newtons;
c. and cooling the resulting fluoropolymer-thermoplastic polymeric composite filter structure.

In a twelfth aspect, the disclosure provides the process of the eleventh aspect, wherein the fluoropolymer is a poly(tetrafluoroethylene).

In a thirteenth aspect, the disclosure provides the process of the eleventh or twelfth aspects, wherein the first polymeric layer is comprised of a polyester.

In a fourteenth aspect, the disclosure provides the process of the eleventh, twelfth, or thirteenth aspects, wherein the second polymeric layer is comprised of a polyester.

In a fifteenth aspect, the disclosure provides the process of any one of the eleventh through the fourteenth aspects, wherein the first polymeric layer is comprised of a poly(ethylene terephthalate) having a melting point from about 95° to about 180° C.

In a sixteenth aspect, the disclosure provides the process of any one of the eleventh through the fifteenth aspects, wherein the second polymeric layer is comprised of a poly(terephthalate) having a melting point from about 220° to about 265° C.

In a seventeenth aspect, the disclosure provides the process of any one of the eleventh through the fifteenth aspects, wherein the first polymeric layer is a poly(ethylene terephthalate) having a melting point from about 150° to about 180° C.

In an eighteenth aspect, the disclosure provides the process of any one of the eleventh through the seventeenth aspects, wherein the fluoropolymer-thermoplastic polymeric composite filter structure exhibits an air flux from about 0.125 to about 0.175 liters/minute and a bonding strength greater than or equal to about 0.35 MPa.

In a nineteenth aspect, the disclosure provides the process of any one of the eleventh through the seventeenth aspects, wherein the fluoropolymer-thermoplastic polymeric composite filter structure exhibits an air flux from about 0.175 to about 210 liters/minute and a bonding strength greater than or equal to about 0.2 MPa.

In a twentieth aspect, the disclosure provides an in-line vent filter device comprising the filter of any one of the first through the tenth aspects.

Having thus described several illustrative embodiments of the present disclosure, those of skill in the art will readily appreciate that yet other embodiments may be made and used within the scope of the claims hereto attached. Numerous advantages of the disclosure covered by this document have been set forth in the foregoing description. It will be understood, however, that this disclosure is, in many respects, only illustrative. The disclosure's scope is, of course, defined in the language in which the appended claims are expressed.

What is claimed is:

1. A filter comprising:
(i) a layer comprising a fluoropolymer membrane; and
(ii) a layer comprising at least two air-permeable thermoplastic polymeric layers, the air-permeable thermoplastic polymeric layers comprised of a first polymeric layer and a second polymeric layer,
wherein the first polymeric layer is in bonded contact with the fluoropolymer membrane and possesses a melting point from about 95° C. to about 180° C., and
wherein the second polymeric layer is in bonded contact with the first polymeric layer and has a melting point from about 220° C. to about 265° C.

2. The filter of claim 1, wherein the filter exhibits an air flux from about 0.125 to about 0.175 liters/minute and a bonding strength greater than or equal to about 0.35 MPa.

3. The filter of claim 1, wherein the filter exhibits an air flux from about 0.175 to about 0.210 liters/minute and a bonding strength greater than or equal to about 0.2 MPa.

4. The filter of claim 1, wherein the air-permeable thermoplastic polymeric layers are comprised of nonwoven fibers.

5. The filter of claim 1, wherein the fluoropolymer membrane is comprised of poly(tetrafluoroethylene).

6. The filter of claim 1, wherein the thermoplastic polymeric layers are comprised of polymers chosen from polyesters and polyolefins.

7. A filter comprising:
(i) a layer comprising at least one poly(tetrafluoroethylene) membrane; and
(ii) a layer comprising at least two polyester nonwoven layers, the polyester nonwoven layers comprised of a first polyester layer and a second polyester layer,
wherein the first polyester layer is in bonded contact with the poly(tetrafluoroethylene) membrane and possesses a melting point from about 95° C. to about 180° C., and wherein the second polyester layer is in bonded contact with the first polyester layer and has a melting point from about 220° C. to about 265° C.

8. The filter of claim 7, wherein the filter exhibits an air flux from about 0.125 to about 0.175 liters/minute and a bonding strength greater than or equal to about 0.35 MPa.

9. The filter of claim 7, wherein the first and second polyester layers are comprised of about 70 to about 100 weight percent of poly(ethylene terephthalate).

10. The filter of claim 7, wherein the melting point of the first polyester is from about 150 to about 180° C.

11. A process for laminating (i) a fluoropolymer membrane and (ii) a bonded layer, the process comprising:
a. applying the bonded layer to the fluoropolymer membrane, wherein the bonded layer comprises at least two air-permeable thermoplastic polymeric layers, the air-permeable thermoplastic polymeric layers comprised of a first polymeric layer having a melting point from about 95° to about 180° C., and a second polymeric layer having a melting point from about 220° to about 265° C., thereby contacting the first polymeric layer of the bonded layer with the fluoropolymer membrane, on a surface having a temperature from about 50° to about 260° C. to form a combined bonded layer and fluoropolymer structure; and
b. subjecting the combined bonded layer and fluoropolymer structure to a temperature of about 140° C. to about 210° C., while compressing at a pressure from about 0.05 to about 0.4 MPa, and while exerting an elongation tension on the combined bonded layer and fluoropolymer structure from about 0.05 to about 0.3 Newtons to form a composite fluoropolymer-thermoplastic polymeric filter structure;
c. and cooling the resulting fluoropolymer-thermoplastic polymeric composite filter structure.

12. The process of claim 11, wherein the fluoropolymer is a poly(tetrafluoroethylene).

13. The process of claim 11, wherein the first polymeric layer is comprised of a polyester

14. The process of claim 11, wherein the second polymeric layer is comprised of a polyester.

15. The process of claim 11, wherein the first polymeric layer is comprised of a poly(ethylene terephthalate) having a melting point from about 95° to about 180° C.

16. The process of claim 11, wherein the second polymeric layer is comprised of a poly(terephthalate) having a melting point from about 220° to about 265° C.

17. The process of claim 11, wherein the first polymeric layer is a poly(ethylene terephthalate) having a melting point from about 150° to about 180° C.

18. The process of claim 11, wherein the fluoropolymer-thermoplastic polymeric composite filter structure exhibits an air flux from about 0.125 to about 0.175 liters/minute and a bonding strength greater than or equal to about 0.35 MPa.

19. The process of claim 11, wherein the fluoropolymer-thermoplastic polymeric composite filter structure exhibits an air flux from about 0.175 to about 0.210 liters/minute and a bonding strength greater than or equal to about 0.2 MPa.

20. An in-line vent filter device comprising the filter of claim 1.
